(19) United States
(12) Patent Application Publication
(10) Pub. No.: US 2024/0379229 A1
(43) Pub. Date: Nov. 14, 2024

(54) CANCER DIAGNOSIS AND CANCER-TYPE PREDICTION METHOD BASED ON CELL-FREE DNA AND IMAGE ANALYSIS TECHNOLOGY

(71) Applicants: GC GENOME CORPORATION, Gyeonggi-do (KR); THE ASAN FOUNDATION, Seoul (KR); UNIVERSITY OF ULSAN FOUNDATION FOR INDUSTRY COOPERATION, Ulsan (KR)

(72) Inventors: CHANG-SEOK KI, Gyeonggi-do (KR); EUN HAE CHO, Gyeonggi-do (KR); JUNNAM LEE, Gyeonggi-do (KR); JIN MO AHN, Gyeonggi-do (KR); SOOK RYUN PARK, Seoul (KR)

(21) Appl. No.: 18/564,601

(22) PCT Filed: May 30, 2022
(86) PCT No.: PCT/KR2022/007661

§ 371 Date: Nov. 27, 2023

(30) Foreign Application Priority Data
May 28, 2021 (KR) 10-2021-0068892

(51) Int. Cl. G16H 50/20; C12Q 1/6806; C40B 50/06; G16B 30/10; G16B 40/20; G16H 30/40

(52) U.S. Cl. CPC G16H 50/20; C12Q 1/6806; C40B 50/06; G16B 30/10; G16B 40/20; G16H 30/40

(57) ABSTRACT

The present invention relates to a method of diagnosing cancer and predicting cancer type using cell-free nucleic acid fragments and image analysis technology, and more particularly, to a method of diagnosing cancer and predicting cancer type by extracting nucleic acids from a biological sample to obtain sequence information (reads), aligning the obtained reads, generating an image including size and coverage information of nucleic acid fragments based on the aligned reads, and then analyzing values calculated by inputting the image into a trained artificial intelligence model. The method of diagnosing cancer and predicting cancer type using size and coverage information of cell-free nucleic acid fragments according to the present invention advantageously shows high sensitivity and accuracy because it generates vectorized data and performs analysis using an AI algorithm.

### TECHNICAL FIELD

The present invention relates to a method of diagnosing cancer and predicting cancer type using cell-free nucleic acid fragments and image analysis technology, and more particularly, to a method of diagnosing cancer and predicting cancer type by extracting nucleic acids from a biological sample to obtain sequence information (reads), aligning the obtained reads, generating an image including size and coverage information of nucleic acid fragments based on the aligned reads, and then analyzing values calculated by inputting the image into a trained artificial intelligence model.

### BACKGROUND ART

Cancer diagnosis in clinical practice is usually performed by tissue biopsy after history examination, physical examination, and clinical evaluation. Cancer diagnosis based on clinical trials is possible only when the number of cancer cells is 1 billion or more and the diameter of the cancer is 1 cm or more. In this case, cancer cells already have the potential to metastasize and at least half thereof have already metastasized. In addition, tissue biopsy is invasive, which disadvantageously causes patients considerable discomfort and is often incompatible with cancer therapy. Further, tumor markers for monitoring substances produced directly or indirectly from cancer are used in cancer screening. However, the tumor markers have limited accuracy because more than half of tumor marker screening results indicate normal even in the presence of cancer and tumor marker screening results often indicate positive even in the absence of cancer.

Recently, in response to the need for relatively simple, non-invasive, highly sensitive, and highly specific cancer diagnosis methods that can overcome the problems of conventional cancer diagnosis methods, liquid biopsy using bodily fluids from patients has been widely used for cancer diagnosis and follow-up examination. Liquid biopsy is a non-invasive diagnostic method that is attracting great attention as an alternative to conventional invasive diagnostic and testing methods.

Recently, methods for cancer diagnosis and cancer-type differentiation using cell-free DNA obtained from liquid biopsy have been developed (US 2020-0219587; WO2020-094775; Zhou, Xionghui et al., bioRxiv, 2020.07.16.201350).

Meanwhile, artificial neural networks are computational models implemented in software or hardware that mimic the computational ability of biological systems using a large number of artificial neurons connected via connective lines. Artificial neural networks use artificial neurons, which represent the functions of biological neurons in simplified form. Artificial neural networks conduct human cognition or learning processes by interconnecting the artificial neurons through connective lines having respective connection intensities. The term “connection intensity”, which is interchangeable with “connection weight”, refers to a predetermined value of the connection line. Artificial neural network learning may be classified into supervised learning and unsupervised learning. Supervised learning is a method of providing input data and output data corresponding thereto to a neural network and updating the connection intensities of connecting lines so that output data corresponding to the input data is output. Representative learning algorithms include delta rule and back propagation learning. Unsupervised learning is a method in which an artificial neural network independently learns connection intensities using only input data, without a target value. Unsupervised learning updates connection weights based on correlations between input patterns.

Applying large amounts of data to machine learning causes the so-called “curse of dimensionality” problem due to the increased complexity and the greater number of dimensions. In other words, as the number of dimensions of the required data approaches infinity, the distance between any two points also approaches infinity, and the amount of data, that is, the density, becomes lower in high-dimensional space, which makes it impossible to properly reflect the features of the data (Richard Bellman, Dynamic Programming, 2003, chapter 1). Recently developed deep learning has a structure in which a hidden layer is present between an input layer and an output layer, and has been reported to greatly improve the performance of the classifier in high-dimensional data such as images, videos, and signal data by processing a linear combination of variable values transmitted from the input layer with nonlinear functions (Hinton, Geoffrey, et al., IEEE Signal Processing Magazine Vol. 29.6, pp. 82-97, 2012).

Various patents (KR 10-2017-0185041, KR 10-2017-0144237, and KR 10-2018-124550) describe the use of artificial neural networks in biological fields, but there is a lack of research on methods of predicting cancer type through artificial neural network analysis based on sequencing information of cell-free DNA (cfDNA).

Accordingly, the present inventors have made extensive efforts to solve the above-described problems and develop an artificial intelligence-based method of diagnosing cancer and predicting cancer type with high sensitivity and accuracy, and as a result, have found that, when an image including size and coverage information of cell-free nucleic acid fragments is generated and analysis of the image is performed using a trained artificial intelligence model, it is possible to diagnose cancer and predict cancer type with high sensitivity and accuracy, thereby completing the present invention.

### Summary of the Invention

An object of the present invention is to provide a method of diagnosing cancer and predicting cancer type using size and coverage information of cell-free nucleic acid fragments.

Another object of the present invention is to provide a system for diagnosing cancer and predicting cancer type using size and coverage information of cell-free nucleic acid fragments.

Still another object of the present invention is to provide a computer-readable storage medium including an instruction configured to be executed by a processor for diagnosing cancer and predicting cancer type by the above-described method.

To achieve the above objects, the present invention provides a method of providing information for diagnosing cancer and predicting cancer type, the method including steps of: (a) obtaining a sequence information by extracting nucleic acids from a biological sample; (b) aligning the obtained sequence information (reads) to a reference genome database; (c) generating an image including size and coverage information of nucleic acid fragments using the aligned sequence information (reads); (d) comparing output values, obtained by inputting the generated image into a trained artificial intelligence model and analyzing the image, with a reference value (cut-off value), thereby determining the presence or absence of cancer; and (e) predicting cancer type through comparison of the output values.

The present invention also provides a system for diagnosing cancer and predicting cancer type, the system including: a decoder configured to extract nucleic acids from a biological sample and decode sequence information; an aligner configured to align the decoded sequence information to a reference genome database; an image generator configured to generate an image including size and coverage information of nucleic acid fragments using the aligned sequence information (reads); a cancer diagnostic unit configured to determine the presence or absence of cancer by inputting the generated image into a trained artificial intelligence model, analyzing the image, and comparing the resulting output value with a reference value (cut-off value); and a cancer type predictor configured to predict cancer type by analyzing the output values.

The present invention also provides a computer-readable storage medium including an instruction configured to be executed by a processor for diagnosing cancer and predicting cancer type through steps of: (a) obtaining a sequence information by extracting nucleic acids from a biological sample; (b) aligning the obtained sequence information (reads) to a reference genome database; (c) generating an image including size and coverage information of nucleic acid fragments using the aligned sequence information (reads); (d) comparing output values, obtained by inputting the generated image into a trained artificial intelligence model and analyzing the image, with a reference value (cut-off value), thereby determining the presence or absence of cancer; and (e) predicting cancer type through comparison of the output values.

The present invention also provides a method for diagnosing cancer and predicting cancer type, the method including steps of: (a) obtaining a sequence information by extracting nucleic acids from a biological sample; (b) aligning the obtained sequence information (reads) to a reference genome database; (c) generating an image including size and coverage information of nucleic acid fragments using the aligned sequence information (reads); (d) comparing output values, obtained by inputting the generated image into a trained artificial intelligence model and analyzing the image, with a reference value (cut-off value), thereby determining the presence or absence of cancer; and (e) predicting cancer type through comparison of the output values.

### BRIEF DESCRIPTION OF DRAWINGS

FIG. 1 is an overall flowchart showing a method of diagnosing cancer and predicting cancer type using size and coverage information of cell-free nucleic acid fragments according to the present invention.

FIG. 2 shows an FSI plot generated in one example of the present invention.

FIG. 3 shows an FSI plot generated in one example of the present invention.

FIG. 4(A) shows the results of analyzing the performance of a CNN model, constructed with a CSI plot generated in one example of the present invention, in an augmented state, and FIG. 4(B) shows the ROC curve of the model (left panel) and the distribution of DPI values (right panel).

FIG. 5(A) shows the results of analyzing the performance of a CNN model, constructed with a CSI plot generated in one example of the present invention, at the sample level, and FIG. 5(B) shows the ROC curve of the model (left panel) and the distribution of DPI values (right panel).

FIG. 6(A) shows the results of analyzing the performance of a CNN model, constructed with an FS plot generated in one example of the present invention, in an augmented state, and FIG. 6(B) shows the ROC curve of the model (left panel) and the distribution of DPI values (right panel).

FIG. 7(A) shows the results of analyzing the performance of a CNN model, constructed with a CSI plot generated in one example of the present invention, at the sample level, and FIG. 7(B) shows the ROC curve of the model (left panel) and the distribution of DPI values (right panel).

FIG. 8 is a schematic view showing the configuration of an FS plot-based CNN model constructed in one example of the present invention.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present invention pertains. In general, the nomenclature used in the present specification and the experimental methods described below are well known and commonly used in the art.

Terms such as first, second, A, B, etc. may be used to describe various components, but the components should not be limited by these terms. These terms are only used for the purpose of distinguishing one component from other components. For example, a first component may be termed a second component without departing from the scope of the present invention, and similarly, a second component may also be termed a first component. The term “and/or” includes any of a plurality of related listed items or a combination of a plurality of related listed items.

In the present specification, singular expressions include plural expressions unless specified otherwise in the context thereof, and terms “include”, etc., are intended to denote the existence of mentioned characteristics, numbers, steps, operations, components, parts, or combinations thereof, but do not exclude the probability of existence or addition of one or more other characteristics, numbers, steps, operations, components, parts, or combinations thereof.

Prior to describing the drawings in detail, it is to be understood that the division of components in the present specification is merely a division according to the main function set for each component. That is, two or more components to be described below may be combined into one component, or one component may be divided into two or more components according to more detailed functions. In addition, it is to be understood that each component to be described below may additionally perform, in addition to the main function thereof, some or all of functions set for other components, and some of the main functions set for each component may be performed by other components.

In addition, when performing a method or an operation method, steps of the method may occur differently from the described order unless a specific order is clearly stated in the context. That is, two consecutively described steps may be performed substantially at the same time or performed in an order opposite to the described order.

In the present invention, it was found that, when sequencing data obtained from a sample were aligned to a reference genome, and then an image including size and coverage information of nucleic acid fragments was generated based on the aligned sequence information, and then a DPI value was calculated using a trained artificial intelligence model, followed by analysis, it was possible to predict cancer and predict a cancer type predicted with high sensitivity and accuracy.

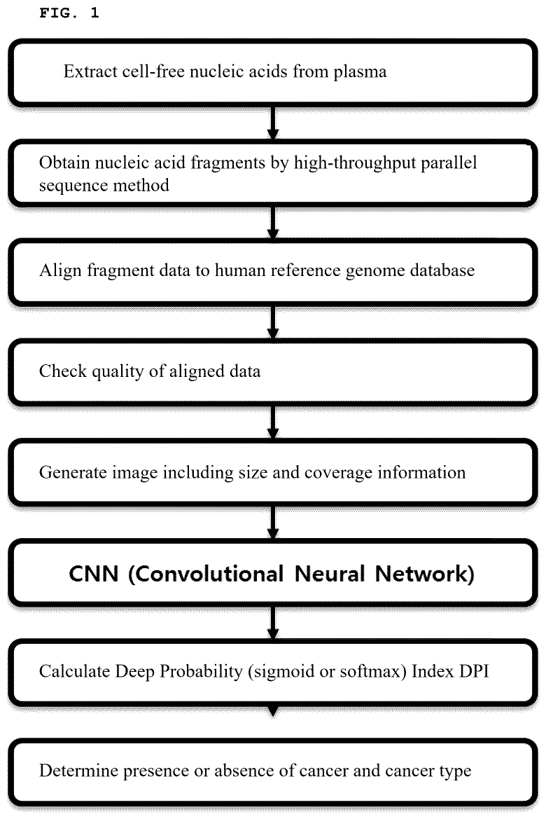

In other words, in one example of the present invention, the present inventors have developed a method including sequencing DNA extracted from blood, aligning the sequence information to a reference genome database, generating an image including size and coverage information of nucleic acid fragments using the aligned sequence information, training a deep learning model for the image to calculate a DPI value, performing cancer diagnosis by comparing the DPI value with a cut-off value, and then determining that the cancer type with the highest DPI value among the DPI values calculated for each cancer type is the cancer type of the sample (FIG. 1).

Therefore, in one aspect, the present invention is directed to a method of providing information for diagnosing cancer and predicting cancer type, the method including steps of:
(a) obtaining a sequence information by extracting nucleic acids from a biological sample;
(b) aligning the obtained sequence information (reads) to a reference genome database;
(c) generating an image including size and coverage information of nucleic acid fragments using the aligned sequence information (reads);
(d) comparing output values, obtained by inputting the generated image into a trained artificial intelligence model and analyzing the image, with a reference value (cut-off value), thereby determining the presence or absence of cancer; and
(e) predicting cancer type through comparison of the output values.

In the present invention, any nucleic acid fragment may be used without limitation, as long as it is a fragment of a nucleic acid extracted from a biological sample. Preferably, the nucleic acid fragment may be a fragment of a cell-free nucleic acid or an intracellular nucleic acid, without being limited thereto.

In the present invention, the nucleic acid fragment may be obtained by any method known to those skilled in the art. Preferably, the nucleic acid fragment may be obtained by direct sequencing, next-generation sequencing, sequencing through non-specific whole-genome amplification, or probe-based sequencing, without being limited thereto.

In the present invention, the nucleic acid fragment may refer to a read when next-generation sequencing is used.

In the present invention, the cancer may be solid cancer or blood cancer. Preferably, the cancer may be selected from the group consisting of non-Hodgkin lymphoma, Hodgkin lymphoma, acute myeloid leukemia, acute lymphoid leukemia, multiple myeloma, head and neck cancer, lung cancer, glioblastoma, colorectal/rectal cancer, pancreatic cancer, breast cancer, ovarian cancer, melanoma, prostate cancer, thyroid cancer, gastric cancer, gallbladder cancer, biliary tract cancer, bladder cancer, small intestine cancer, cervical cancer, cancer of unknown primary origin, kidney cancer, esophageal cancer, and mesothelioma. More preferably, the cancer may be liver cancer or esophageal cancer, without being limited thereto.

In the present invention,
step (a) may include:
   (a-i) collecting nucleic acids from a biological sample;
   (a-ii) purifying purified nucleic acids by removing proteins, fats, and other residues from the collected nucleic acids using a salting-out method, a column chromatography method, or a bead-based method;
   (a-iii) constructing a single-end sequencing or paired-end sequencing library for either the purified nucleic acids or nucleic acids randomly fragmented by an enzymatic cleavage, atomization, or hydroshear method;
   (a-iv) subjecting the constructed library to a next-generation sequencer; and
   (a-v) obtaining sequence information (reads) of the nucleic acids from the next-generation sequencer.

In the present invention, step (a) of obtaining sequence information may include obtaining acquiring isolated cell-free DNA through whole-genome sequencing at a depth of 1 million to 100 million reads.

In the present invention, the biological sample refers to any substance, biological fluid, tissue or cell obtained from or derived from a subject, and examples thereof include, but are not limited to, whole blood, leukocytes, peripheral blood mononuclear cells, buffy coat, blood (including plasma and serum), sputum, tears, mucus, nasal washes, nasal aspirate, breath, urine, semen, saliva, peritoneal washings, pelvic fluids, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, semen, hair, saliva, urine, oral cells, placental cells, cerebrospinal fluid, and mixtures thereof.

In the present invention, the next-generation sequencer may employ any sequencing method known in the art. Sequencing of nucleic acids isolated using the selection method is typically performed using next-generation sequencing (NGS). Next-generation sequencing includes any sequencing method that determines the nucleotide sequence of either individual nucleic acid molecules (e.g., in single molecule sequencing) or clonally expanded proxies for individual nucleic acid molecules in a highly parallel fashion (e.g., greater than 105 molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, which is incorporated herein by reference.

In one embodiment, the next-generation sequencing allows for the determination of the nucleotide sequence of an individual nucleic acid molecule (e.g., Helicos BioSciences' HeliScope Gene Sequencing system, and Pacific Biosciences' PacBio RS system). In other embodiments, the sequencing method determines the nucleotide sequence of clonally expanded proxies for individual nucleic acid molecules (e.g., the Solexa sequencer, Illumina Inc., San Diego, Calif; 454 Life Sciences (Branford, Conn.), and Ion Torrent), e.g., massively parallel short-read sequencing (e.g., the Solexa sequencer, Illumina Inc., San Diego, Calif.), which generates more bases of sequence per sequencing unit than other sequencing methods that generate fewer but longer reads. Other methods or machines for next-generation sequencing include, but not limited to, the sequencers provided by 454 Life Sciences (Branford, Conn.), Applied Biosystems (Foster City, Calif.; SOLID sequencer), Helicos BioSciences Corporation (Cambridge, Mass.), and emulsion and microfluidic sequencing technology nanodroplets (e.g., GnuBio droplets).

Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLID) system, Polonator's G.007 system, BioSciences' HeliScope Gene Sequencing system, and Pacific Biosciences' PacBio RS system.

NGS technologies can include one or more of steps, e.g., template preparation, sequencing and imaging, and data analysis.

Template preparation. Methods for template preparation can include steps such as randomly breaking nucleic acids (e.g., genomic DNA or cDNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates.

Methods for preparing clonally amplified templates include, e.g., emulsion PCR (emPCR) and solid-phase amplification.

EmPCR can be used to prepare templates for NGS. Typically, a library of nucleic acid fragments is generated, and adaptors containing universal priming sites are ligated to the ends of the fragment. The fragments are then denatured into single strands and captured by beads. Each bead captures a single nucleic acid molecule. After amplification and enrichment of emPCR beads, a large amount of templates can be attached or immobilized in a polyacrylamide gel on a standard microscope slide (e.g., Polonator), chemically crosslinked to an amino-coated glass surface (e.g., Life/APG; Polonator), or deposited into individual PicoTiterPlate (PTP) wells (e.g., Roche/454), in which the NGS reaction can be performed.

Solid-phase amplification can also be used to produce templates for NGS. Typically, forward and reverse primers are covalently attached to a solid support. The surface density of the amplified fragments is defined by the ratio of the primers to the templates on the support. Solid-phase amplification can produce hundreds of millions of spatially separated template clusters (e.g., Illumina/Solexa). The ends of the template clusters can be hybridized to universal sequencing primers for NGS reactions.

Other methods for preparing clonally amplified templates also include, e.g., Multiple Displacement Amplification (MDA) (Lasken R. S. Curr Opin Microbiol. 2007; 10(5): 510-6). MDA is a non-PCR based DNA amplification technique. The reaction involves annealing random hexamer primers to the template and DNA synthesis by high-fidelity enzyme, typically Φ29 at a constant temperature. MDA can generate large sized products with lower error frequency.

Template amplification methods such as PCR can be coupled with NGS platforms to target or enrich specific regions of the genome (e.g., exons). Exemplary template enrichment methods include, e.g., microdroplet PCR technology (Tewhey R. et al., Nature Biotech. 2009, 27:1025-1031), custom-designed oligonucleotide microarrays (e.g., Roche/NimbleGen oligonucleotide microarrays), and solution-based hybridization methods (e.g., molecular inversion probes (MIPs) (Porreca G. J. et al., Nature Methods, 2007, 4:931-936; Krishnakumar S. et al., Proc. Natl. Acad. Sci. USA, 2008, 105:9296-9310; Turner E. H. et al., Nature Methods, 2009, 6:315-316), and biotinylated RNA capture sequences (Gnirke A. et al., Nat. Biotechnol. 2009; 27 (2): 182-9).

Single-molecule templates are another type of template that can be used for NGS reaction. Spatially separated single molecule templates can be immobilized on solid supports by various methods. In one approach, individual primer molecules are covalently attached to the solid support. Adaptors are added to the templates and templates are then hybridized to the immobilized primers. In another approach, single-molecule templates are covalently attached to the solid support by priming and extending single-stranded, single-molecule templates from immobilized primers. Universal primers are then hybridized to the templates. In yet another approach, single polymerase molecules are attached to the solid support, to which primed templates are bound.

Sequencing and imaging. Exemplary sequencing and imaging methods for NGS include, but are not limited to, cyclic reversible termination (CRT), sequencing by ligation (SBL), single-molecule addition (pyrosequencing), and real-time sequencing.

CRT uses reversible terminators in a cyclic method that minimally includes the steps of nucleotide incorporation, fluorescence imaging, and cleavage. Typically, a DNA polymerase incorporates a single fluorescently modified nucleotide corresponding to the complementary nucleotide of the template base to the primer. DNA synthesis is terminated after the addition of a single nucleotide and the unincorporated nucleotides are washed away. Imaging is performed to determine the identity of the incorporated labeled nucleotide. Then in the cleavage step, the terminating/inhibiting group and the fluorescent dye are removed. Exemplary NGS platforms using the CRT method include, but are not limited to, Illumina/Solexa Genome Analyzer (GA), which uses the clonally amplified template method coupled with the four-color CRT method detected by total internal reflection fluorescence (TIRF); and Helicos BioSciences/HeliScope, which uses the single-molecule template method coupled with the one-color CRT method detected by TIRF.

SBL uses DNA ligase and either one-base-encoded probes or two-base-encoded probes for sequencing.

Typically, a fluorescently labeled probe is hybridized to its complementary sequence adjacent to the primed template. DNA ligase is used to ligate the dye-labeled probe to the primer. Fluorescence imaging is performed to determine the identity of the ligated probe after non-ligated probes are washed away. The fluorescent dye can be removed by using cleavable probes to regenerate a 5′-PO₄ group for subsequent ligation cycles. Alternatively, a new primer can be hybridized to the template after the old primer is removed. Exemplary platforms include, without being limited to, Life/APG/SOLID (support oligonucleotide ligation detection), which uses two-base-encoded probes.

Pyrosequencing method is based on detecting the activity of DNA polymerase using another chemiluminescent enzyme. Typically, the method allows sequencing of a single strand of DNA by synthesizing the complementary strand along it, one base pair at a time, and detecting which base was actually added at each step. The template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template. Exemplary pyrosequencing platforms include, but are not limited to, Roche/454, which uses DNA templates prepared by emPCR with 1-2 million beads deposited into PTP wells.

Real-time sequencing involves imaging the continuous incorporation of dye-labeled nucleotides during DNA synthesis. Exemplary real-time sequencing platforms include, but are not limited to, the Pacific Biosciences platform, which uses DNA polymerase molecules attached to the surface of individual zero-mode waveguide (ZMW) detectors to obtain sequence information when phospholinked nucleotides are being incorporated into the growing primer strand; the Life/VisiGen platform, which uses an engineered DNA polymerase with an attached fluorescent dye to generate an enhanced signal after nucleotide incorporation by fluorescence resonance energy transfer (FRET); and the LI-COR Biosciences platform, which uses dye-quencher nucleotides in the sequencing reaction.

Other sequencing methods for NGS include, but are not limited to, nanopore sequencing, sequencing by hybridization, nano-transistor array based sequencing, Polony sequencing, scanning tunneling microscopy (STM) based sequencing, and nanowire-molecule sensor based sequencing.

Nanopore sequencing involves electrophoresis of nucleic acid molecules in solution through a nano-scale pore which provides a highly confined space within which single-nucleic acid polymers can be analyzed. Exemplary methods of nanopore sequencing are described, e.g., in Branton D. et al., Nat Biotechnol. 2008; 26 (10): 1146-53.

Sequencing by hybridization is a non-enzymatic method that uses a DNA microarray. Typically, a single pool of DNA is fluorescently labeled and hybridized to an array containing known sequences. Hybridization signals from a given spot on the array can identify the DNA sequence. The binding of one strand of DNA to its complementary strand in the DNA double-helix is sensitive to even single-base mismatches when the hybrid region is short or when specialized mismatch detection proteins are present. Exemplary methods of sequencing by hybridization are described, e.g., in Hanna G. J. et al., J. Clin. Microbiol. 2000; 38 (7): 2715-21; and Edwards J. R. et al., Mut. Res. 2005; 573 (1-2): 3-12.

Polony sequencing is based on Polony amplification and sequencing-by-synthesis via multiple single-base-extensions (FISSEQ). Polony amplification is a method to amplify DNA in situ on a polyacrylamide film. Exemplary Polony sequencing methods are described, e.g., in US Patent Application Publication No. 2007/0087362.

Nano-transistor array based devices, such as Carbon NanoTube Field Effect Transistors (CNTFETs), can also be used for NGS. For example, DNA molecules are stretched and driven over nanotubes by micro-fabricated electrodes. DNA molecules sequentially come into contact with the carbon nanotube surface, and the difference in current flow from each base is produced due to charge transfer between the DNA molecule and the nanotubes. DNA is sequenced by recording these differences. Exemplary Nano-transistor array based sequencing methods are described, e.g., in U.S. Patent Application Publication No. 2006/0246497.

Scanning tunneling microscopy (STM) can also be used for NGS. STM uses a piezoelectric-controlled probe that performs a raster scan of a specimen to form images of its surface. STM can be used to image the physical properties of single DNA molecules, e.g., generating coherent electron tunneling imaging and spectroscopy by integrating a scanning tunneling microscope with an actuator-driven flexible gap. Exemplary sequencing methods using STM are described, e.g., in U.S. Patent Application Publication No. 2007/0194225.

A molecular-analysis device which is comprised of a nanowire-molecule sensor can also be used for NGS. Such device can detect the interactions of the nitrogenous material disposed on the nanowires and nucleic acid molecules such as DNA. A molecule guide is configured to guide a molecule near the molecule sensor, allowing an interaction and subsequent detection. Exemplary sequencing methods using nanowire-molecule sensor are described, e.g., in U.S. Patent Application Publication No. 2006/0275779.

Double ended sequencing methods can be used for NGS. Double ended sequencing uses blocked and unblocked primers to sequence both the sense and antisense strands of DNA. Typically, these methods include the steps of annealing an unblocked primer to a first strand of nucleic acid; annealing a second blocked primer to a second strand of nucleic acid; elongating the nucleic acid along the first strand with a polymerase; terminating the first sequencing primer; deblocking the second primer; and elongating the nucleic acid along the second strand. Exemplary double ended sequencing methods are described, e.g., in U.S. Pat. No. 7,244,567.

After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild-type sequence). Methods of sequence alignment for NGS are described e.g., in Trapnell C. and Salzberg S. L. Nature Biotech., 2009, 27:455-457.

Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829.

Sequence alignment or assembly can be performed using read data from one or more NGS platforms, e.g., mixing Roche/454 and Illumina/Solexa read data. In the present invention, the alignment step may be performed using the BWA algorithm and the hg19 sequence, without being limited thereto.

In the present invention, sequence alignment in step (b) includes a computational method or approach used to identify from where in the genome a read sequence (e.g., a short-read sequence, e.g., from next-generation sequencing) most likely originated by assessing the similarity between the read sequence and a reference sequence. A variety of algorithms can be applied to the sequence alignment problem. Some algorithms are relatively slow, but allow relatively high specificity. These include, e.g., dynamic programming-based algorithms. Dynamic programming is a method for solving complex problems by breaking them down into simpler steps. Other approaches are relatively more efficient, but are typically not as thorough. These include, e.g., heuristic algorithms and probabilistic methods designed for large-scale database search.

Typically, there can be two steps in the alignment process: candidate lookup and sequence alignment. Candidate lookup reduces the search space for sequence alignment from the entire genome to a shorter list of possible alignment locations. Sequence alignment, as the term suggests, includes aligning a sequence with a sequence provided in a candidate lookup step. Sequence alignment can be performed using global alignment (e.g., Needleman-Wunsch alignment) or local alignment (e.g., Smith-Waterman alignment).

Most of fast alignment algorithms can be characterized as one of the following three types based on the method of indexing: algorithms based on hash tables (e.g., BLAST, ELAND, SOAP), suffix trees (e.g., Bowtie, BWA), and merge sorting (e.g., Slider). Short read sequences are typically used for alignment. Examples of sequence alignment algorithms/programs for short-read sequences include, but are not limited to, BFAST (Homer N. et al., PLOS One. 2009; 4 (11): e7767), BLASTN (on the worldwide web atblast.ncbi.nlm.nih.gov), BLAT (Kent W. J. Genome Res. 2002; 12 (4): 656-64), Bowtie (Langmead B. et al., Genome Biol. 2009; 10 (3): R25), BWA (Li H. and Durbin R. Bioinformatics, 2009, 25:1754-60), BWA-SW (Li H. and Durbin R. Bioinformatics, 2010; 26 (5): 589-95), CloudBurst (Schatz M. C. Bioinformatics. 2009; 25 (1 1): 1363-9), Corona Lite (Applied Biosystems, Carlsbad, California, USA), CASHX (Fahlgren N. et al., RNA, 2009; 15, 992-1002), CUDA-EC (Shi H. et al., J ComputBiol. 2010; 17 (4): 603-15), ELAND (on the worldwide web atbioit.dbi.udel.edu/howto/eland), GNUMAP (Clement N. L. et al., Bioinformatics. 2010; 26 (1): 38-45), GMAP (Wu T. D. and Watanabe C. K. Bioinformatics. 2005; 21 (9): 1859-75), GSNAP (Wu T. D. and Nacu S, Bioinformatics. 2010; 26 (7): 873-81), Geneious Assembler (Biomatters Ltd., Auckland, New Zealand), LAST, MAQ (Li H. et al., Genome Res. 2008; 18 (1 1): 1851-8), Mega-BLAST (on the worldwide web at ncbi.nlm.nih.gov/blast/megablast.shtml), MOM (Eaves H. L. and Gao Y. Bioinformatics. 2009; 25 (7): 969-70), MOSAIK (on the worldwide web at bioinformatics.bc.edu/marthlab/Mosaik), Novoalign (on the worldwide web at novocraft.com/main/index.php), PALMapper (on the worldwide web at fml.tuebingen.mpg.de/raetsch/suppl/palmapper), PASS (Campagna D. et al., Bioinformatics. 2009; 25 (7): 967-8), PatMaN (Prufer K. et al., Bioinformatics. 2008; 24 (13): 1530-1), PerM (Chen Y. et al., Bioinformatics, 2009, 25 (19): 2514-2521), ProbeMatch (Kim Y. J. et al., Bioinformatics. 2009; 25 (1 1): 1424-5), QPalma (de Bona F. et al., Bioinformatics, 2008, 24 (16): H74), RazerS (Weese D. et al., Genome Research, 2009, 19:1646-1654), RMAP (Smith A. D. et al., Bioinformatics. 2009; 25 (21): 2841-2), SeqMap (Jiang H. et al. Bioinformatics. 2008; 24:2395-2396.), Shrec (Salmela L, Bioinformatics. 2010; 26 (10): 1284-90), SHRIMP (Rumble S. M. et al., PLOS Comput. Biol., 2009, 5 (5): e1000386), SLIDER (Malhis N. et al., Bioinformatics, 2009, 25 (1): 6-13), SLIM Search (Muller T. et al., Bioinformatics. 2001; 17 Suppl 1: S182-9), SOAP (Li R. et al., Bioinformatics. 2008; 24 (5): 713-4), SOAP2 (Li R. et al., Bioinformatics. 2009; 25 (15): 1966-7), SOCS (Ondov B. D. et al., Bioinformatics, 2008; 24 (23): 2776-7), SSAHA (Ning Z. et al., Genome Res. 2001; 11 (10): 1725-9), SSAHA2 (Ning Z. et al., Genome Res. 2001; 11 (10): 1725-9), Stampy (Lunter G. and Goodson M. Genome Res. 2010, epub ahead of print), Taipan (on the worldwide web at taipan.sourceforge.net), UGENE (on the worldwide web at ugene.unipro.ru), XpressAlign (on the worldwide web at bcgsc.ca/platform/bioinfo/software/XpressAlign), and ZOOM (Bioinformatics Solutions Inc., Waterloo, ON, Canada).

A sequence alignment algorithm can be chosen based on a number of factors including, e.g., the sequencing technology, read length, number of reads, available compute resources, and sensitivity/scoring requirements. Different sequence alignment algorithms can achieve different levels of speed, alignment sensitivity, and alignment specificity. Alignment specificity typically refers to the percentage of aligned target sequence residues, as found in the submission, which are aligned correctly, compared with the predicted alignment. Alignment sensitivity usually refers to the percentage of aligned target sequence residues as found in the predicted alignment, which have also been correctly aligned in the submission.

Alignment algorithms, such as ELAND, or SOAP can be used for the purpose of aligning short reads (e.g., from Illumina/Solexa sequencer) to the reference genome when speed is the first factor to consider. Alignment algorithms, such as BLAST, or Mega-BLASTcan be used for the purpose of similarity search using short reads (e.g., from Roche FLX) when specificity is the most important factor, although these methods are relatively slow. Alignment algorithms, such as MAQ, or Novoalign take quality scores into account and therefore can be used for both single or paired-end data sets when accuracy is of the essence (e.g., in high-throughput SNP surveys). Alignment algorithms, such as Bowtie, or BWA, use Burrows-Wheeler Transform (BWT) and therefore require a relatively small memory footprint. Alignment algorithms, such as BFAST, PerM, SHRIMP, SOCS, or ZOOM, map color space reads and therefore can be used with ABI's SOLID platform. In some applications, the results from two or more alignment algorithms can be combined.

In the present invention, the length of the sequence information (reads) in step (b) may be 5 to 5,000 bp, and the number of sequence information (reads) that are used may be 5,000 to 5 million, without being limited thereto.

In the present invention, the image including size and coverage information of nucleic acid fragments in step (c) may be a CSI plot (Coverage and Size Information plot) or a FS plot (Fragment Size plot), without being not limited thereto.

In the present invention, the method may further include, before step (c), a step of separately classifying nucleic acid fragments that satisfy the mapping quality score of the aligned nucleic acid fragments.

In the present invention, the mapping quality score may vary depending on a desired standard, but is preferably 15 to 70, more preferably 50 to 70, most preferably 60.

In the present invention, the CSI plot may be generated by a method including steps of:
(i) dividing chromosomes into predetermined bins;
(ii) determining the number of nucleic acid fragments aligned in each bin; and
iii) generating a CSI plot with the order of respective bins on the X-axis and a value obtained by classifying the value determined in step (ii) by the size of the nucleic acid fragment on the Y-axis.

In the present invention, the FS plot may be generated by a method including steps of:
(i) dividing chromosomes into predetermined bins;
(ii) determining the number of nucleic acid fragments aligned in each bin;
(iii) classifying the determined number of nucleic acid fragments by the size of the nucleic acid fragment;
(iv) performing normalization by dividing the value calculated in step (iii) by the value determined in step ii);
(v) generating a plot with the order of respective bins on the X-axis and the normalized value calculated in step (iv) on the Y-axis; and
(vi) generating a FS plot by stacking the plots, generated for the respective chromosomes, based on image channels.

Usually, an image is in the form of a 2×2 matrix, and if colors are included, RGB color channels are required, and thus there are three 2×2 matrices (representing R, G, and B, respectively). Thus, in the present invention, the expression “stacking the plots based on image channels” means stacking each image as if it represents R, G, and B channels.

In the present invention, the normalization step may be performed using Equation 3 below.

Equation 3: Normalized value NCij=Cij/Σⱼ₌₁₀₀²⁰⁰Cij wherein, Cij means the number of fragments with length j in the i^{th} bin.

In the present invention, the bin may be used without limitation as long as it has a fixed size value. The fixed size value is preferably 500 kbp, 1 Mbp, 5 Mbp, or the like, without being limited thereto.

In the present invention, the size of the nucleic acid fragment may be the number of bases from the 5′ end to the 3′ end of the nucleic acid fragment. In the present invention, the size of the nucleic acid fragment may be 1 to 10,000 bp, preferably 10 to 1,000 bp, more preferably 50 to 500 bp, most preferably 90 to 250 bp, without being limited thereto.

In the present invention, in the step of classifying the number of the nucleic acid fragments aligned in each bin by the size of the nucleic acid fragment, the size of each nucleic acid fragment may be set according to the required purpose. For example, the number of the nucleic acid fragments aligned in each bin may be classified by dividing the size of each nucleic acid fragment in units of bp, such as 90 bp, 91 bp, 92 bp . . . , 198 bp, 199 bp, 200 bp, or the like.

In the present invention, as the artificial intelligence model in step (d), any model may be used without limitation as long as it is a model that can learn to distinguish between images for each cancer type. Preferably, the artificial intelligence model is a deep learning model.

In the present invention, as the artificial intelligence model, an artificial neural network algorithm capable of analyzing images based on an artificial neural network may be used without limitation. Preferably, the artificial intelligence model may be selected from the group consisting of a convolutional neural network (CNN), a deep neural network (DNN), and a recurrent neural network (RNN), without being limited thereto.

In the present invention, the recurrent neural network may be selected from the group consisting of a long-short term memory (LSTM) neural network, a gated recurrent unit (GRU) neural network, a vanilla recurrent neural network, and an attentive recurrent neural network.

In the present invention, when the artificial intelligence model is a CNN, the loss function for performing binary classification may be represented by Equation 1 below, and the loss function for performing multi-class classification may be represented by Equation 2 below.

Equation 1: Binary classification

$$loss ( model ( x ) , y ) = - \frac{1}{n} [ {\sum }_{i = 1}^{n} ( {y}_{i} log ( model ( {x}_{i} ) ) + ( 1 - {y}_{i} ) log ( 1 - model ( {x}_{i} ) ) ) ]$$

model(xᵢ)=artificial intelligence model output in response to i^{th} input
y=actual label value
n=number of input data

Equation 2: Multi-class classification

$$loss ( model ( x ) , y ) = - \frac{1}{n} {\sum }_{i = 1}^{n} ( {\sum }_{j = 1}^{c} ( {y}_{ij} {log ( model ( {x}_{i} ) )}_{j} )$$

model(xᵢ)ⱼ=j^{th} artificial intelligence model output in response to i^{th} input
y=actual label value
n=number of input data
c=number of classes

In the present invention, the binary classification means that the artificial intelligence model learns to identify the presence or absence of cancer, and the multi-class classification means that the artificial intelligence model learns to distinguish between two or more cancer types.

In the present invention, when the artificial intelligence model is a CNN, learning may include the following steps:
i) classifying the produced vector data into training, validation, and test data,
wherein the training data are used when the CNN model is trained, the validation data are used for hyper-parameter tuning validation, and the test data are used for performance evaluation after optimal model production;
ii) constructing an optimal CNN model through hyper-parameter tuning and training; and
iii) comparing the performance of multiple models, obtained through hyper-parameter tuning, using validation data, and determining that the model having the best validation data is the optimal model.

In the present invention, hyper-parameter tuning is a process of optimizing the values of various parameters (the number of convolution layers, the number of dense layers, the number of convolution filters, etc.) constituting the CNN model. Hyper-parameter tuning may be performed using Bayesian optimization and grid search techniques.

In the present invention, the internal parameters (weights) of the CNN model may be optimized using predetermined hyper-parameters, and it may be determined that the model is over-fit when validation loss starts to increase compared to training loss. Training of the model may be stopped prior to over-fitting.

In the present invention, any value resulting from analysis of the input vectorized data by the artificial intelligence model in step (d) may be used without limitation, as long as it is a specific score or real number. Preferably, the value may be a deep probability index (DPI), without being limited thereto.

In the present invention, “deep probability index” means a value expressed as a probability value by adjusting the output of artificial intelligence to a scale of 0 to 1 using, for the last layer of the artificial intelligence model, a sigmoid function in the case of binary classification and a SoftMax function in the case of multi-class classification.

In binary classification, training is performed using the sigmoid function such that the DPI is 1 when the sample is cancer. For example, when a breast cancer sample and a normal sample are input, training is performed such that the DPI of the breast cancer sample is close to 1.

In multi-class classification, as many DPIs as the number of classes are extracted using the SoftMax function. The sum of the DPIs is adjusted to 1 and training is performed such that the DPI value of the actually corresponding cancer type becomes 1. For example, provided that there are three classes, namely, breast cancer, liver cancer, and normal classes, when a breast cancer sample is input, training is performed such that the DPI value of the breast cancer class is close to about 1.

In the present invention, the output value resulting from step (d) may be obtained for each cancer type.

In the present invention, the artificial intelligence model is trained such that the output value is close to 1 if there is cancer and such that the output value is close to 0 if there is no cancer. Therefore, performance (training, validation, and test accuracy) is measured based on a cut-off value of 0.5. In other words, if the output value is 0.5 or more, it is determined that there is cancer, and if the output value is less than 0.5, it is determined that there is no cancer.

Here, it will be apparent to those skilled in the art that the cut-off value of 0.5 may be arbitrarily changed. For example, in order to reduce false positives, the cut-off value may be set to be higher than 0.5 as a stricter criterion for determining that there is cancer, and in order to reduce false negatives, the cut-off value may be set to be lower than 0.5 as a weaker criterion for determining that there is cancer.

Most preferably, the cut-off value may be set by checking the probability of the DPI by applying unseen data (data for which the answer is not trained for learning data) using the trained artificial intelligence model.

In the present invention, step (e) of predicting cancer type through comparison of the output values may be performed by a method including a step of determining that a cancer type showing the highest value among the output values is the cancer of the sample.

In another aspect, the present invention is directed to a system for diagnosing cancer and predicting cancer type,
the system including:
   a decoder configured to extract nucleic acids from a biological sample and decode sequence information;
   an aligner configured to align the decoded sequence information to a reference genome database;
   an image generator configured to generate an image including size and coverage information of nucleic acid fragments using the aligned sequence information (reads);
   a cancer diagnostic unit configured to determine the presence or absence of cancer by inputting the generated image into a trained artificial intelligence model, analyzing the image, and comparing the resulting output values with a reference value (cut-off value); and
   a cancer type predictor configured to predict cancer type by analyzing the output values.

In the present invention, the decoder may include a nucleic acid input unit configured to input nucleic acids extracted using an independent device, and a sequence information analyzer configured to analyze sequence information of the input nucleic acid. Preferably, the decoder may be an NGS analyzer, without being limited thereto.

In the present invention, the decoder may receive and decode the sequence information data generated in the independent device.

In another aspect, the present invention is directed to a computer-readable storage medium including an instruction configured to be executed by a processor for diagnosing cancer and predicting cancer type through steps of:
(a) obtaining a sequence information by extracting nucleic acids from a biological sample;
(b) aligning the obtained sequence information (reads) to a reference genome database;
(c) generating an image including size and coverage information of nucleic acid fragments using the aligned sequence information (reads);
(d) comparing output values, obtained by inputting the generated image into a trained artificial intelligence model and analyzing the image, with a reference value (cut-off value), thereby determining the presence or absence of cancer; and
(e) predicting cancer type through comparison of the output values.

In another aspect, the method according to the present invention may be implemented using a computer. In one embodiment, the computer includes one or more processors coupled to a chipset. In addition, a memory, a storage device, a keyboard, a graphics adapter, a pointing device, a network adapter and the like are connected to the chipset. In one embodiment, the performance of the chipset is acquired by a memory controller hub and an I/O controller hub. In another embodiment, the memory may be directly coupled to a processor instead of the chipset. The storage device is any device capable of maintaining data, including a hard drive, compact disc read-only memory (CD-ROM), DVD, or other memory devices. The memory is involved data and instructions used by the processor. The pointing device may be a mouse, track ball or other type of pointing device, and is used in combination with a keyboard to transmit input data to a computer system. The graphics adapter presents images and other information on a display. The network adapter is connected to the computer system through a local area network or a long distance communication network. However, the computer used herein is not limited to the above configuration, may not have some configurations, may further include additional configurations, and may also be part of a storage area network (SAN), and the computer of the present invention may be configured to be suitable for the execution of modules in the program for the implementation of the method according to the present invention.

As used herein, the term “module” may refer to a functional and structural combination of hardware to implement the technical idea according to the present invention and software to drive the hardware. For example, it will be apparent to those skilled in the art that the module may refers to a logical unit of a predetermined code and a hardware resource for executing the predetermined code, and does not necessarily mean a physically connected code or one type of hardware.

In another aspect, the present invention is directed to a method for diagnosing cancer and predicting cancer type, the method including steps of:
(a) obtaining a sequence information by extracting nucleic acids from a biological sample;
(b) aligning the obtained sequence information (reads) to a reference genome database;
(c) generating an image including size and coverage information of nucleic acid fragments using the aligned sequence information (reads);
(d) comparing output values, obtained by inputting the generated image to a trained artificial intelligence model and analyzing the image, with a reference value (cut-off value), thereby determining the presence or absence of cancer; and
(e) predicting cancer type through comparison of the output values.

### EXAMPLES

Hereinafter, the present invention will be described in more detail with reference to examples. However, these examples are only for illustrating the present invention, and it will be obvious to those skilled in the art that the scope of the present invention should not be construed as being limited by these examples.

### Example 1. Extraction of DNA from Blood and Carrying Out of Next-Generation Sequencing

10 mL of blood was collected from each of 350 normal subjects, 51 hepatocellular carcinoma patients, and 108 esophageal cancer patients, and stored in an EDTA tube. Within 2 hours after blood collection, only plasma was collected by first centrifugation at 1,200 g at 4° C. for 15 minutes, and then the collected plasma was subjected to second centrifugation at 16,000 g at 4° C. for 10 minutes, thereby isolating the plasma supernatant excluding the precipitate. Cell-free DNA was extracted from the isolated plasma using a Tiangen Micro DNA kit (Tiangen) and prepared into libraries using a TruSeq Nano DNA HT library prep kit (Illumina), and then sequencing was performed in 100-base paired-end mode using DNBseq G400 (MGI). As a result, it was confirmed that about 170 million reads were produced per sample.

### Example 2. Generation of CSI Plot Image

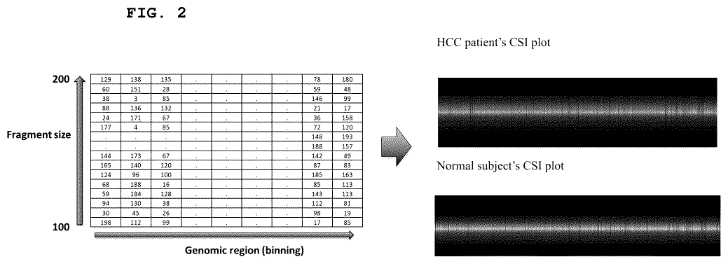

A CSI plot was generated using the NGS data generated in Example 1 above. Specifically, the entire chromosome was divided into bins of 2 megabase pairs, and set on the X-axis, and the size of the nucleic acid fragment was set on the Y-axis. The number of nucleic acid fragments counted for each nucleic acid fragment size in each bin was plotted, thereby generating a CSI plot in the form of a heatmap (FIG. 2).

### Example 3. CNN Model Construction and Training Process

Using the CSI plot as input, a CNN artificial intelligence model was trained to distinguish between normal subjects, hepatocellular carcinoma (HCC) patients, and esophageal cancer (EC) patients.

To prevent overfitting and improve model reliability, all samples were down-sampled about 10 times to generate images, followed by construction of models (augmentation). All data were divided into training, validation, and test groups. Models were constructed using the training samples, and the performance of the models generated using the training samples was evaluated using the samples of the validation and test groups. The number of samples for each set is as follows.

TABLE 1

| type | total | HCC | EC | Normal | | type | total | HCC | EC | Normal |
|---|---|---|---|---|---|---|---|---|---|---|
| Train | 267 | 13 | 60 | 194 | [symbol] | Train | 2358 | 108 | 529 | 1721 |
| Valid | 119 | 26 | 22 | 71 | ~×10 | Valid | 1051 | 223 | 196 | 632 |
| Test | 123 | 12 | 26 | 85 | | Test | 1093 | 107 | 225 | 761 |
| Total | 509 | 51 | 108 | 350 | | Total | 4502 | 438 | 950 | 3114 |

Hyper-parameter tuning is a process of optimizing the values of various parameters (the number of convolution layers, the number of dense layers, the number of convolution filters, etc.) constituting the CNN model. Hyper-parameter tuning was performed using Bayesian optimization and grid search techniques, and when validation loss started to increase compared to training loss, the model was determined to be over-fit, and training of the model was stopped.

The performance of various models obtained through hyper-parameter tuning was compared using the validation data set, and then the model having the best validation data set performance was determined to be the optimal model, and final performance evaluation was performed using the test data set.

When the CSI plot image of any sample is input into the model constructed through the above-described process, the probability of the sample being a normal subject, the probability of the sample being an HCC patient, and the probability of the sample being an esophageal cancer patient are calculated through the SoftMax function, which is the last layer of the CNN model. These probability values were defined as deep probability index (DPI) values.

Any sample is determined to be the group having the highest value among the three types of DPI values. For example, if the DPI values for a normal subject, a HCC patient, and an esophageal cancer patient, calculated from any sample, were 0.6, 0.3, and 0.1, respectively, this sample would be determined to be a normal subject.

### Example 4. Evaluation of Performance of Constructed Deep-Learning Model

### 4-1. Evaluation of Performance in Augmented State

The performance of the DPI values output from the deep-learning model constructed in Example 3 was tested.

TABLE 2

| | Training | Validation | Test |
|---|---|---|---|
| Accuracy | 0.973 | 0.865 | 0.897 |
| Micro AUC | 0.998 | 0.961 | 0.969 |
| Macro AUC | 1.000 | 0.943 | 0.964 |

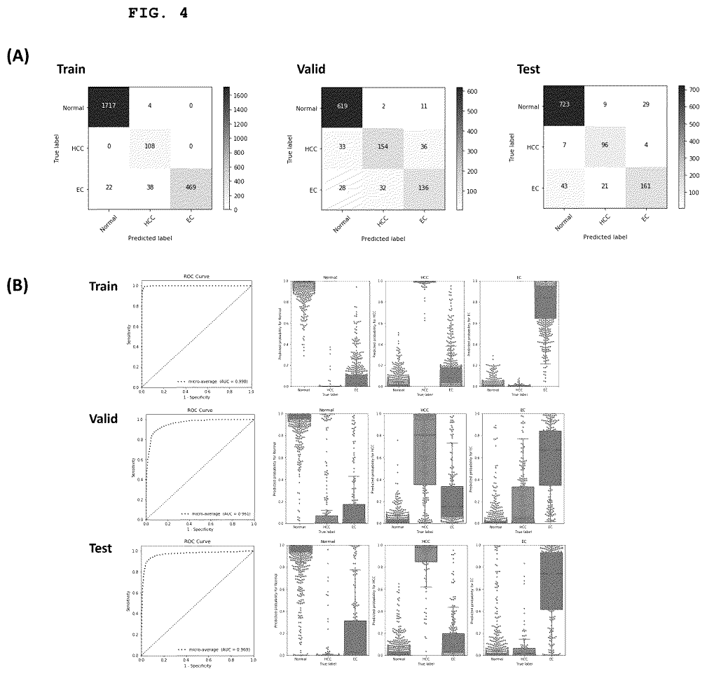

As a result, as shown in Table 2 above and FIG. 4, it was confirmed that accuracies were 0.973, 0.865, and 0.897 in the training, validation, and test groups, respectively, and that the AUC values, which indicate the results of ROC analysis, were 0.998, 0.961, and 0.970 in the training, validation, and test groups, respectively, and Macro AUC 0.943, and 0.964 in the training, values were 1.000, validation, and test groups, respectively.

The right panel in FIG. 4(B) shows the results of analyzing the distribution of DPI values output from the constructed model. In the right panel, the X-axis represents group (true label) information of the actual sample, and the Y-axis represents the DPI values for normal subjects (Normal), hepatocellular carcinoma (HCC) patients, and esophageal cancer (EC) patients in order from the left, calculated from the CNN model.

As shown in FIG. 4, the distribution of DPI values indicated that, in all of training, validation, and test data sets, the normal samples had the highest probability of being normal subjects, the HCC patient samples had the highest probability of being HCC patients, and the esophageal cancer patient samples had the highest probability of being esophageal cancer patients.

### 4-2. Evaluation of Performance at Sample Level

As a result of the augmentation analysis in Example 4-1 above, about 10 DPI values were calculated from one sample. The median value of the distribution of these values was defined as the DPI value of the sample, and analysis was performed based on this value in the same manner as in Example 4-1.

TABLE 3

| | Training | Validation | Test |
|---|---|---|---|
| Accuracy | 0.985 | 0.856 | 0.926 |
| Macro AUC | 0.998 | 0.949 | 0.972 |
| Micro AUC | 0.999 | 0.967 | 0.975 |

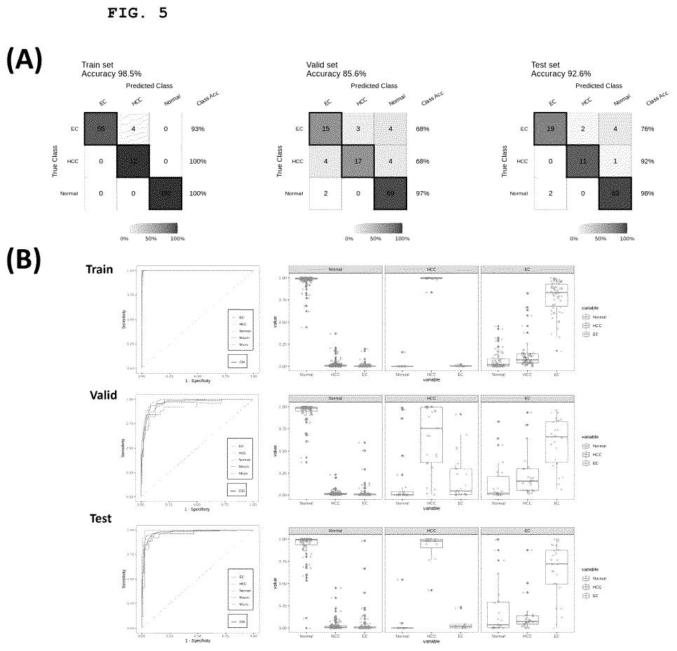

As a result, as shown in Table 3 above and FIG. 5, it was confirmed that accuracies were 0.985, 0.856, and 0.926 in the training, validation, and test groups, respectively, the macro AUC values, which are the results of ROC analysis, were 0.998, 0.949, and 0.972 in the training, validation, and test groups, respectively, and the micro AUC values were 0.999, 0.967, and 0.975, in the training, validation, and test groups, respectively.

The right panel in FIG. 5(B) shows the results of analyzing the distribution of DPI values output from the constructed model. In the right panel, the X-axis represents group (true label) information of the actual sample, and the Y-axis represents the DPI values for normal subjects (Normal), cancer (EC) patients in order from the left, calculated from the CNN model.

As shown in FIG. 5, the distribution of DPI values indicated that, in all of training, validation, and test data sets, the normal samples had the highest probability of being normal subjects, the HCC patient samples had the highest probability of being HCC patients, and the esophageal cancer patient samples had the highest probability of being esophageal cancer patients.

### Example 5. Generation of FS Plot Image

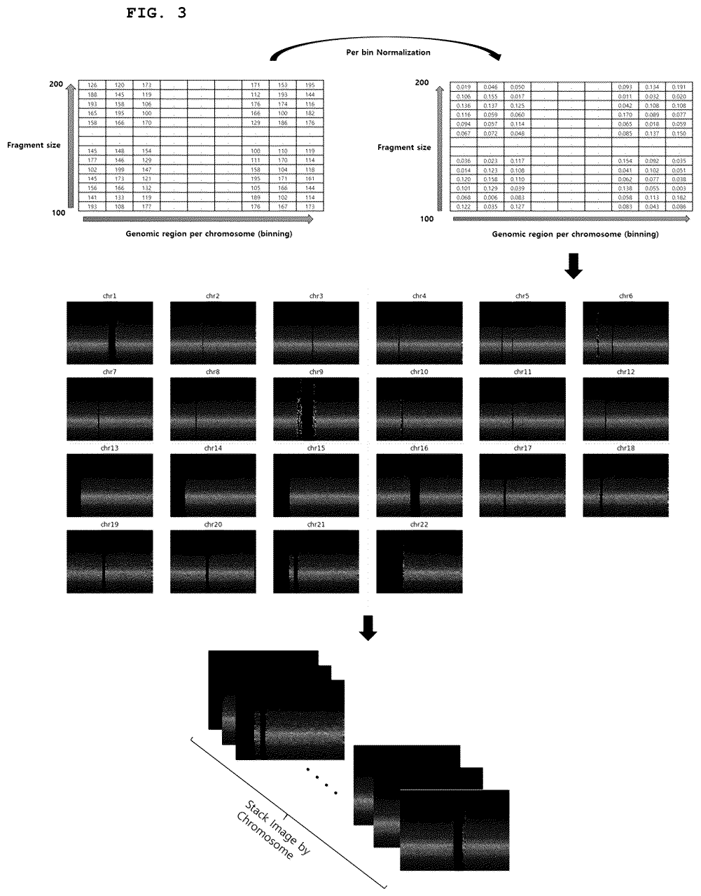

A FS plot image was generated using the NGS data generated in Example 1 above. Specifically, each chromosome was divided into bins of 1,000,000 bp and set on the X-axis, and the size of the nucleic acid fragment was set on the Y-axis. The number of nucleic acid fragments counted for each nucleic acid fragment size in each bin was divided by the total number of nucleic acid fragments corresponding to each bin, and the resulting normalized value was plotted, thereby generating images in the form of a heatmap. These images were stacked based on image channels, thereby generating an FS plot image (FIG. 3).

The size of each stacked image was 100×500×1, and the size of the input image obtained by stacking the images of all chromosomes except sex chromosomes was 100×500×22.

At this time, normalization for each bin was performed by calculation using Equation 3 below.

Equation 3: Normalized value NCij=Cij/Σⱼ₌₁₀₀²⁰⁰Cij wherein, Cij means the number of fragments with length j in the i^{th} bin.

### Example 6. CNN Model Construction and Training Process

Using the FS plot as input, a CNN artificial intelligence model was trained to distinguish between normal subjects, hepatocellular carcinoma (HCC) patients, and esophageal cancer (EC) patients.

To prevent overfitting and improve model reliability, all samples were down-sampled about 10 times to generate images, followed by construction of models (augmentation). All data were divided into training, validation, and test groups. Models were constructed using the training samples, and the performance of the models generated using the training samples was evaluated using the samples of the validation and test groups. The number of samples for each set is as follows.

TABLE 4

| Type | Normal | EC | HCC | Total | | Type | Normal | EC | HCC | Total |
|---|---|---|---|---|---|---|---|---|---|---|
| Train | 193 | 60 | 26 | 279 | [symbol] | Train | 1930 | 600 | 260 | 2790 |
| Valid | 71 | 22 | 12 | 105 | ~×10 | Valid | 710 | 220 | 120 | 1050 |
| Test | 85 | 26 | 13 | 124 | | Test | 850 | 260 | 130 | 1240 |
| Total | 349 | 108 | 51 | 508 | | Total | 3490 | 1080 | 510 | 5080 |

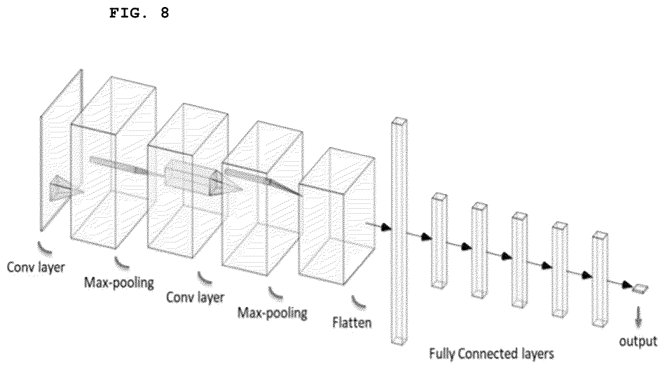

The basic configuration of the CNN model is shown in FIG. 8. As the activation function, ReLU (rectified linear unit) function was used, and as each convolution layer, twenty 10×10 patches were used. The pooling mode used herein was max pooling and 2×2 patches were used. Five fully connected layers were used and each layer included 175 hidden nodes. Finally, the final DPI value was calculated using the sigmoid function value.

Hyper-parameter tuning is a process of optimizing the values of various parameters (the number of convolution layers, the number of dense layers, the number of convolution filters, etc.) constituting the CNN model. Hyper-parameter tuning was performed using Bayesian optimization and grid search techniques, and when validation loss started to increase compared to training loss, the model was determined to be over-fit, and training of the model was stopped.

The performance of various models obtained through hyper-parameter tuning was compared using the validation data set, and then the model having the best validation data set performance was determined to be the optimal model, and final performance evaluation was performed using the test data set.

When the CSI plot image of any sample is input to the model constructed through the above-described process, the probability of the sample being a normal subject, the probability of the sample being an HCC patient, and the probability of the sample being an esophageal cancer patient are calculated through the SoftMax function, which is the last layer of the CNN model. These probability values were defined as deep probability index (DPI) values.

Any sample is determined to be the group having the highest value among the three types of DPI values. For example, if the DPI values for a normal subject, an HCC patient, and an esophageal cancer (EC) patient, calculated from any sample, were 0.6, 0.3, and 0.1, respectively, this sample would be determined to be HCC.

### Example 7. Evaluation of Performance of Constructed Deep-Learning Model

### 7-1. Evaluation of Performance in Augmented State

The performance of the DPI values output from the deep-learning model constructed in Example 6 was tested.

TABLE 5

| | Training | Validation | Test |
|---|---|---|---|
| Accuracy | 1.000 | 0.919 | 0.927 |
| Micro AUC | 1.000 | 0.986 | 0.981 |
| Macro AUC | 1.000 | 0.979 | 0.973 |

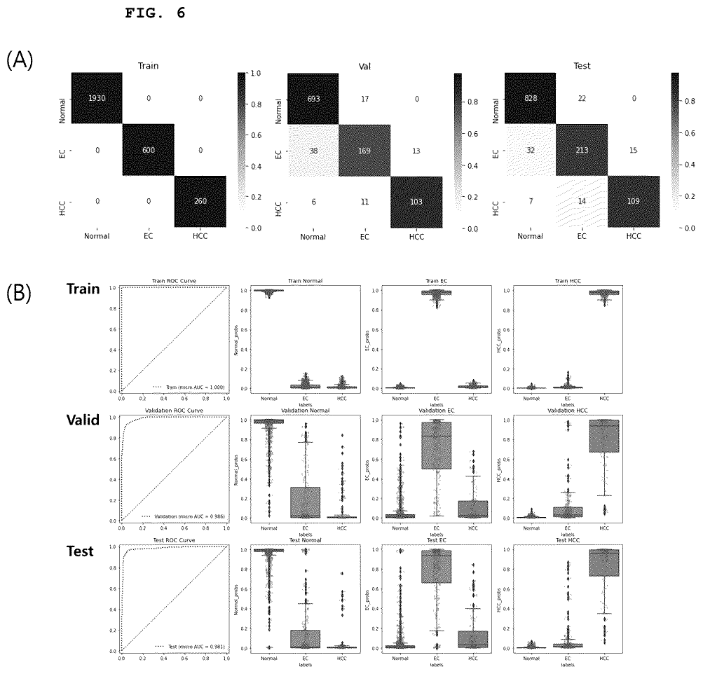

As a result, as shown in Table 5 above and FIG. 6, it was confirmed that accuracies were 1.000, 0.919, and 0.927 in the training, validation, and test groups, respectively, and that the AUC values, which indicate the results of ROC analysis, were 1.000, 0.986, and 0.981 in the training, validation, and test groups, respectively, and Macro AUC values were 1.000, 0.979, and 0.973 in the training, validation, and test groups, respectively.

The right panel in FIG. 6(B) shows the results of analyzing the distribution of DPI values output from the constructed model. In the right panel, the X-axis represents group (true label) information of the actual sample, and the Y-axis represents the DPI values for normal subjects (Normal), hepatocellular carcinoma (HCC) patients, and esophageal cancer (EC) patients in order from the left, calculated from the CNN model.

As shown in FIG. 6, the distribution of DPI values indicated that, in all of training, validation, and test data sets, the normal samples had the highest probability of being normal subjects, the HCC patient samples had the highest probability of being HCC patients, and the esophageal cancer patient samples had the highest probability of being esophageal cancer patients.

### 6-2. Evaluation of Performance at Sample Level

As a result of the augmentation analysis in Example 6-1 above, about 10 DPI values were calculated from one sample. The median value of the distribution of these values was defined as the DPI value of the sample, and analysis was performed based on this value in the same manner as in Example 4-1.

TABLE 6

| | Training | Validation | Test |
|---|---|---|---|
| Accuracy | 1.000 | 0.943 | 0.944 |
| Micro AUC | 1.000 | 0.988 | 0.982 |
| Macro AUC | 1.000 | 0.983 | 0.972 |

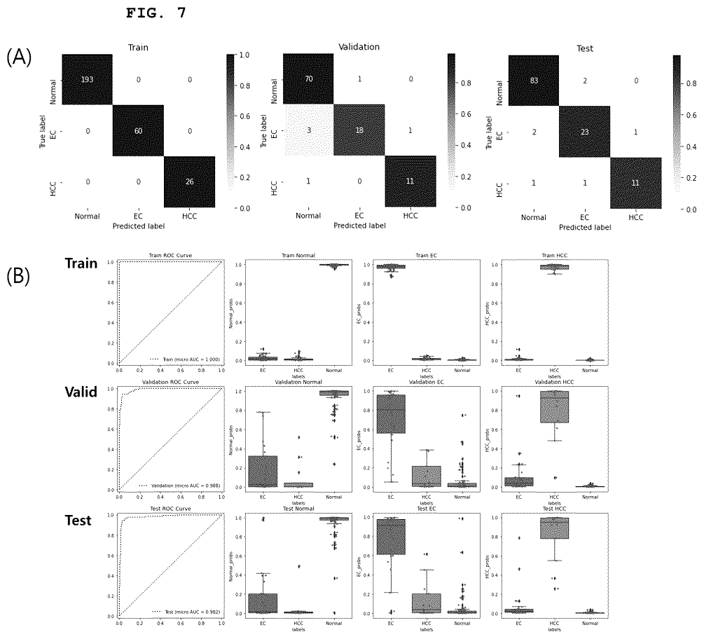

As a result, as shown in Table 6 above and FIG. 7, it was confirmed that accuracies were 1.000, 0.943, and 0.944 in the training, validation, and test groups, respectively, the macro AUC values, which are the results of ROC analysis, were 1.000, 0.988, and 0.982 in the training, validation, and test groups, respectively, and the micro AUC values were 1.000, 0.983, and 0.972 in the training, validation, and test groups, respectively.

The right panel in FIG. 7(B) shows the results of analyzing the distribution of DPI values output from the constructed model. In the right panel, the X-axis represents group (true label) information of the actual sample, and the Y-axis represents the DPI values for normal subjects (Normal), cancer (EC) patients in order from the left, calculated from the CNN model.

As shown in FIG. 7, the distribution of DPI values indicated that, in all of training, validation, and test data sets, the normal samples had the highest probability of being normal subjects, the HCC patient samples had the highest probability of being HCC patients, and the esophageal cancer patient samples had the highest probability of being esophageal cancer patients.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The method of diagnosing cancer and predicting cancer type using size and coverage information of cell-free nucleic acid fragments according to the present invention advantageously shows high sensitivity and accuracy because it generates vectorized data and performs analysis using an AI algorithm.

1. (canceled)

2. A method for diagnosing cancer and predicting cancer type, the method comprising steps of:
(a) obtaining a sequence information by extracting nucleic acids from a biological sample;
(b) aligning the obtained sequence information (reads) to a reference genome database;
(c) generating an image including size and coverage information of nucleic acid fragments using the aligned sequence information (reads);
(d) comparing output values, obtained by inputting the generated image into a trained artificial intelligence model and analyzing the image, with a reference value (cut-off value), thereby determining the presence or absence of cancer; and
(e) predicting cancer type through comparison of the output values.

3. The method according to claim 2, wherein step (a) is performed by a method comprising steps of:
(a-i) collecting nucleic acids from a biological sample;
(a-ii) obtaining purified nucleic acids by removing proteins, fats, and other residues from the collected nucleic acids using a salting-out method, a column chromatography method, or a bead-based method;
(a-iii) constructing a single-end sequencing or paired-end sequencing library for either the purified nucleic acids or nucleic acids randomly fragmented by an enzymatic cleavage, atomization, or hydroshear method;
(a-iv) subjecting the constructed library to a next-generation sequencer; and
(a-v) obtaining sequence information (reads) of the nucleic acids from the next-generation sequencer.

4. The method according to claim 2, wherein the image including size and coverage information of nucleic acid fragments is a CSI plot (Coverage and Size Information plot) or an FS plot (Fragment Size plot).

5. The method according to claim 4, wherein the CSI plot is generated by a method comprising steps of:
(i) dividing chromosomes into predetermined bins;
(ii) determining a number of nucleic acid fragments aligned in each bin; and
iv) generating a CSI plot with the order of respective bins on the X-axis and a value obtained by classifying the value determined in step (ii) by the size of the nucleic acid fragment on the Y-axis.

6. The method according to claim 4, wherein the FS plot is generated by a method comprising steps of:
(i) dividing chromosomes into predetermined bins;
(ii) determining a number of nucleic acid fragments aligned in each bin;
(iii) classifying the determined number of nucleic acid fragments by the size of the nucleic acid fragment;
(iv) performing normalization by dividing the value calculated in step (iii) by the value determined in step ii);
(v) generating a plot with the order of respective bins on the X-axis and the normalized value calculated in step (iv) on the Y-axis; and
(vi) generating an FS plot by stacking the plots, generated for the respective chromosomes, based on image channels.

7. The method according to claim 2, wherein the artificial intelligence model in step (d) learns to distinguish between normal images and images with cancer.

8. The method according to claim 7, wherein the artificial intelligence model is selected from the group consisting of a convolutional neural network (CNN), a deep neural network (DNN), and a recurrent neural network (RNN).

9. The method according to claim 8, wherein, when the artificial intelligence model is the CNN and learns binary classification, a loss function is represented by Equation 1 below, and when the artificial intelligence model is the CNN and learns multi-class classification, a loss function is represented by Equation 2 below.
Equation 1: Binary classification
$$loss ( model ( x ) , y ) = - \frac{1}{n} [ {\sum }_{i = 1}^{n} ( {y}_{i} log ( model ( {x}_{i} ) ) + ( 1 - {y}_{i} ) log ( 1 - model ( {x}_{i} ) ) ) ]$$
model(xᵢ)=artificial intelligence model output in response to i^{th} input
y=actual label value
n=number of input data
Equation 2: Multi-class classification
$$loss ( model ( x ) , y ) = - \frac{1}{n} {\sum }_{i = 1}^{n} ( {\sum }_{j = 1}^{c} ( {y}_{ij} {log ( model ( {x}_{i} ) )}_{j} )$$
model(xᵢ)ⱼ=j^{th} artificial intelligence model output in response to i^{th} input
y=actual label value
n=number of input data
c=number of classes.

10. The method according to claim 2, wherein the output values obtained by inputting the generated image into a trained artificial intelligence model and analyzing the image in step (d) are deep probability index (DPI) values.

11. The method according to claim 2, wherein the cut-off value in step (d) is 0.5, and when the output values are 0.5 or more, the sample is determined to be cancer.

12. The method according to claim 2, wherein step (e) of predicting cancer type through comparison of the output values is performed by a method comprising a step of determining that a cancer type showing the highest value among the output values is the cancer of the sample.

13. A system for diagnosing cancer and predicting cancer type, the system comprising:
a decoder configured to extract nucleic acids from a biological sample and decode sequence information;
an aligner configured to align the decoded sequence information to a reference genome database;
an image generator configured to generate an image including size and coverage information of nucleic acid fragments using the aligned sequence information (reads);
a cancer diagnostic unit configured to determine the presence or absence of cancer by inputting the generated image into a trained artificial intelligence model, analyzing the image, and comparing the resulting output values with a reference value (cut-off value); and
a cancer-type predictor configured to predict cancer type by analyzing the output values.

14. A computer-readable storage medium including an instruction configured to be executed by a processor for diagnosing cancer and predicting cancer type through steps of:
(a) obtaining a sequence information by extracting nucleic acids from a biological sample;
(b) aligning the obtained sequence information (reads) to a reference genome database;
(c) generating an image including size and coverage information of nucleic acid fragments using the aligned sequence information (reads);
(d) comparing output values, obtained by inputting the generated image into a trained artificial intelligence model and analyzing the image, with a reference value (cut-off value), thereby determining the presence or absence of cancer; and
(e) predicting cancer type through comparison of the output values.
